(19) United States
(12) Patent Application Publication
(10) Pub. No.: US 2007/0244432 A1
(43) Pub. Date: Oct. 18, 2007

(54) SYRINGE NEEDLE DEVICE

(76) Inventors: Yung-Wen Shen, Hsinying City (TW); Chih-Chang Huang, Yungho City (TW)

Correspondence Address: KAMRATH & ASSOCIATES P.A., 4825 OLSON MEMORIAL HIGHWAY, SUITE 245, GOLDEN VALLEY, MN, 55422, US

(21) Appl. No.: 11/279,440

(22) Filed: Apr. 12, 2006

(51) Int. Cl. A61M 5/00

(52) U.S. Cl. 604/110

(57) ABSTRACT

A syringe needle device comprises a needle, a ring and a cover, wherein a needle seat of the needle can be engaged with the cover, a stopping portion is mounted on the needle seat for stopping the ring, thereby the needle is retained in the cover after taking out the ring and mounting on the cover.

### BACKGROUND OF THE INVENTION

1. Field of the Invention

The present invention relates to a syringe needle device, which comprises a needle, a ring and a cover, wherein a needle seat of the needle can be engaged with the cover, a stopping portion is mounted on the needle seat for stopping the ring, thus injecting after taking out the ring and the cover, and retaining the needle in the cover by combining the needle seat with the cover directly after injection so as to attain the purpose of safety.

2. Description of the Prior Art

A syringe needle device is the most widely used medical materials, the needles are directly contacted with medicament or patient's blood, so that the used needles have some potentially risks, different patients cannot share the same needle, and the medical persons and the cleaners must be careful so as to avoid pricking by the needles, thereby how to dispose of those needles safely are very important.

A conventional syringe device, the cover is defined with a tapering portion thereof for mounting the needle seat so as to retain the needle portion, however, it is difficult to ensure the needle seat is mounted with the cover stably, that is to say, the strength mounting the cover to the needle seat is not enough, therefore the cover is probable to loose during throwing away or carrying, thus the cleaners are easier to be infected after being pricked by the needles.

The present invention has arisen to mitigate and/or obviate the afore-described disadvantages.

### SUMMARY OF THE INVENTION

The primary objective of the present invention is to provide a syringe needle device, which includes a needle, a ring and a cover, wherein a needle seat of the needle can be engaged with the cover, a stopping portion is mounted on the needle seat for stopping the ring, and the advantages are describes as follows:

Firstly, since the syringe needle device of the present invention includes a needle, a ring and a cover, wherein the ring is mounted on the stopping portion of the needle seat, so that the ring and the cover can be taken out simultaneously when using in order to draw blood or inject the medicament, and after finishing, the medical person can mount the cover on the needle directly for engaging the needle seat with the cover so as to retain the needle in the cover, thus attain the purpose of safety.

Secondly, the syringe needle device of the present invention will not change the original operating method of the medical person, the problem of operating unsuitably will not be happened (at present, some syringe needle devices are used by a special operating method, which is easier to fail because of unsuitable operating, and for the medical persons it is troublesome).

The present invention will become more obvious from the following description when taken in connection with the accompanying drawings, which show, for purpose of illustrations only, the preferred embodiments in accordance with the present invention.

### BRIEF DESCRIPTION OF THE DRAWINGS

FIG. 1 is a structural view of a syringe needle device in accordance with the present invention;

FIG. 2 is a perspective view of showing the syringe needle device combined with a syringe in accordance with the present invention;

FIG. 3 is a cross sectional view of showing a cover covered a needle in accordance with the present invention;

FIG. 4 is an exploded view of the syringe needle device in accordance with the present invention;

FIG. 5 is a further cross sectional view of showing the cover covered the needle in accordance with another embodiment of the present invention;

FIG. 6 is an exploded view of another cover in accordance with the present invention;

FIG. 7 is another perspective view of the cover in accordance with the present invention; and

FIG. 8 is a further perspective view of the cover in accordance with the present invention.

### DETAILED DESCRIPTION OF THE PREFERRED EMBODIMENTS

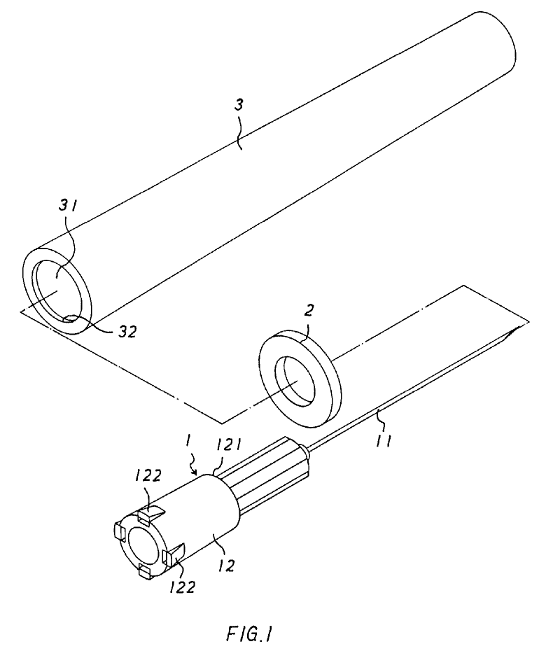

Please refer to FIG. 1, where a syringe needle device 10 in accordance with the present invention comprises a needle 1, a ring 2 and a cover 3, wherein:

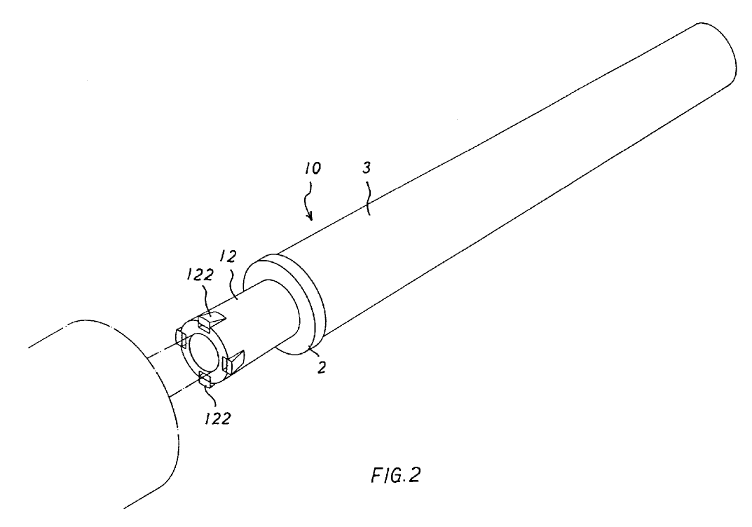

The needle 1, including a needle portion 11 and a needle seat 12 for combining with a syringe, a stopping portion 121 is protrudently formed on the needle seat 12, and the end of the needle seat 12 is protrudently formed more than one elastic engaging hook 122 as shown in FIG. 2.

The ring 2 is mounted on the needle seat 12 and stopped by the stopping portion 121.

The cover 3, provided for retaining the needle 1, including an edge 32 in an opening end 31 of the cover 3 thereof.

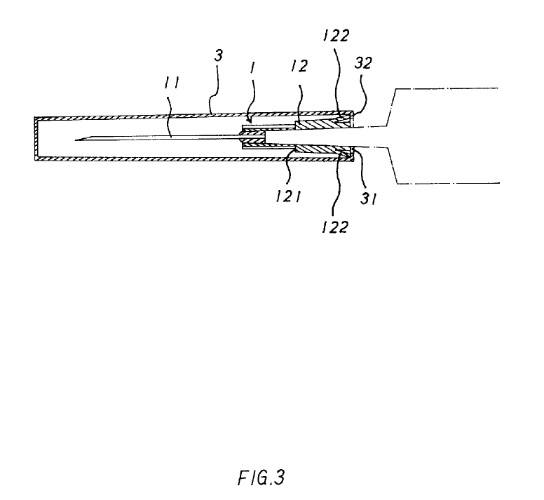

By such arrangement, the needle 1, the ring 2 and the cover 3 of the syringe needle device 10 are mounted orderly in turns (especially the packing of the syringe needle device 10), thereby the cover 3 cannot be engaged with the needle 1 directly before the ring 2 is detached, thus the needle 1 will not be engaged into the cover 3 before the use of the syringe needle device 10. Furthermore, when taking out the syringe needle device 10, the ring 2 and the cover 3 can be taken out simultaneously, at that time, the needle 1 is served for drawing blood or injecting the medicament, and the cover 3 can be mounted on the needle 1 directly so as to deform the elastic engaging hook 122 of the needle seat 12, so that the needle 1 will be retained in the cover 3, and a stopping function can be formed in the cover 3 opposite to the edge 32 as shown in FIG. 3, so as to prevent the needle 1 is taken out from the cover 3 and ensure the needle 1 is retained in the cover 3.

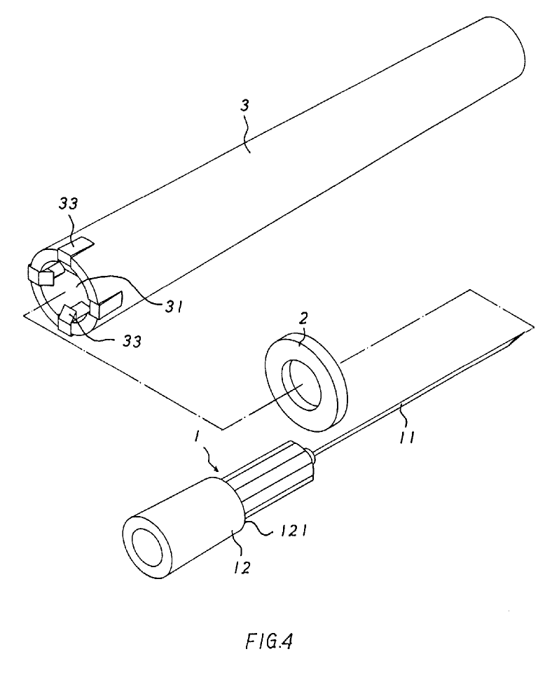

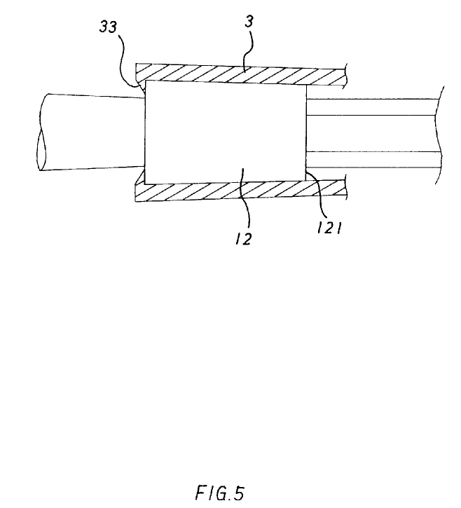

Please refer then to FIG. 4, where the syringe needle device in accordance with another embodiment of the present invention, the opening end 31 of the cover 3 is disposed more than one elastic engaging hook 33, the needle 1 includes the needle portion 11 and the needle seat 12 for combining with a syringe, the stopping portion 121 is protrudently formed on the needle seat 12 for stopping the ring 2. By such arrangement, after being used, the cover 3 is mounted on the needle 1 directly (at that time, the elastic engaging hooks 33 will be deformed outwardly along the needle seat 12) so as to retain the needle 1 in the cover 3, and prevent the needle 1 separating from the cover 3 by the stopping function of the elastic engaging hooks 33 as shown in FIG. 5, thus attaining the purpose of safety.

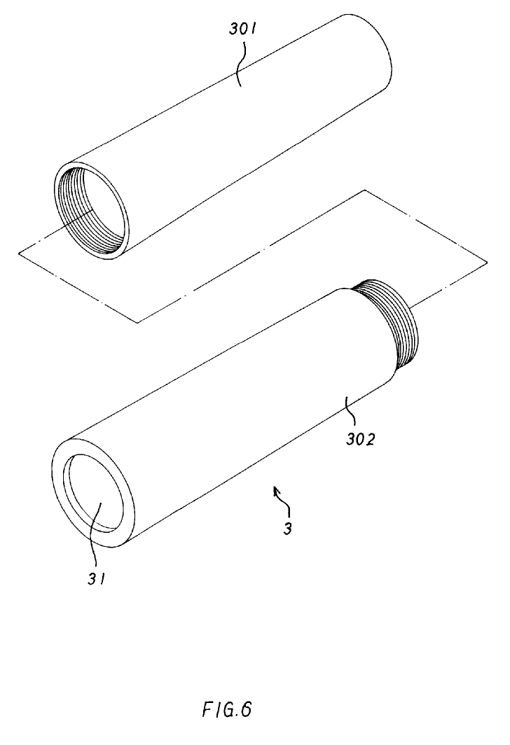

In view of patient's requirement that the medical person cannot draw blood and inject the medicament into the patient once (e.g. the patient felt uncomfortable during the injection), at that time, the needle 1 must be covered, however, in order to make the covered needle 1 used for the same patient again, the cover 3 of the present invention is divided into a front cover 301 and a rear cover 302 that are helically connected to each other as shown in FIG. 6, thereby when taking the front cover 301 out, the needle portion 11 of the needle 1 can be exposed successfully for drawing blood and injecting the medicament into the patient again.

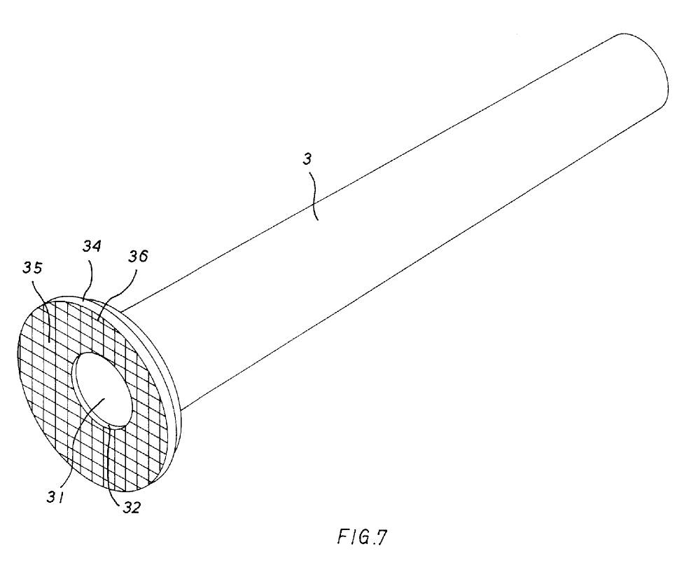

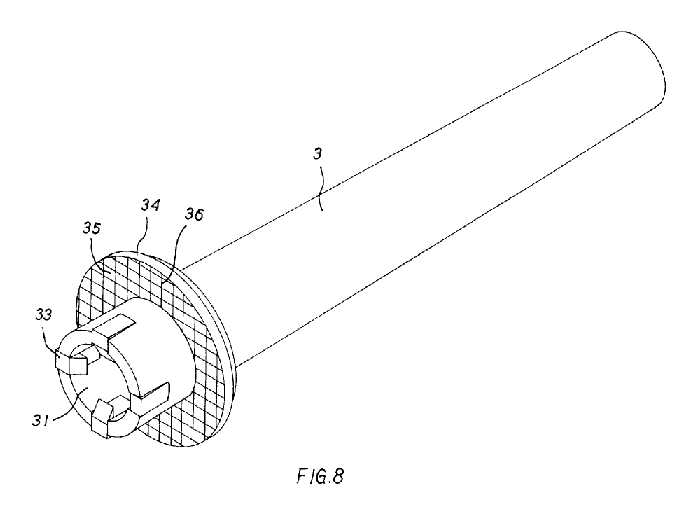

In addition, please refer to FIGS. 7 and 8, where a stopping ring 34 is mounted near the opening end 31 of the cover 3, including a tapering surface 35 towards the opening end 31, and the tapering surface 35 is defined with anti-sliding grains 36 that are served as a stopping surface for protecting the fingers of the medical person, and preventing the needle portion 11 pricked into the fingers by the anti-sliding grains 36 when the needle portion 11 is contacted with the tapering surface 35 accidentally.

While we have shown and described various embodiments in accordance with the present invention, it should be clear to those skilled in the art that further embodiments may be made without departing from the scope of the present invention.

What is claimed is:

1. A syringe needle device, including a needle, a ring and a cover, wherein a needle seat of the needle is engaged with the cover, a stopping portion is mounted on the needle seat for stopping the ring, thereby the needle is retained in the cover after taking out the ring and mounting on the cover.

2. The syringe needle device as claimed in claim 1, wherein the needle includes a needle portion and a needle seat for combining with a syringe, a stopping portion is protrudently formed on the needle seat for stopping the ring, and the end of the needle seat is protrudently formed more than one elastic engaging hook for corresponding to an edge in an opening end of the cover.

3. The syringe needle device as claimed in claim 1, wherein the opening end of the cover is disposed more than one elastic engaging hook, the needle includes the needle portion and the needle seat for combining with a syringe, the stopping portion is protrudently formed on the needle seat for stopping the ring, thereby the needle is retained in the cover and the elastic engaging hook is provided for preventing the needle separated from the cover.

4. The syringe needle device as claimed in claim 1, wherein a stopping ring is mounted near the opening end of the cover.

5. The syringe needle device as claimed in claim 1, wherein a stopping ring defined with a tapering surface towards the opening end is mounted near the opening end of the cover.

6. The syringe needle device as claimed in claim 1, wherein the cover is divided into a front cover and a rear cover that are helically connected to each other.

7. The syringe needle device as claimed in claim 4, wherein the stopping ring is mounted with anti-sliding grains.

8. The syringe needle device as claimed in claim 2, wherein the cover is divided into a front cover and a rear cover that are helically connected to each other.

9. The syringe needle device as claimed in claim 5, wherein the stopping ring is mounted with anti-sliding grains.

10. The syringe needle device as claimed in claim 3, wherein the cover is divided into a front cover and a rear cover that are helically connected to each other.
